SEQUENCE LISTING (47 sequences)

SEQ ID NO: 1 - DNA, 49 bp, Homo sapiens
gggataagaagctaaccaaagcccatgtgttcgagtgtaatttagagag

SEQ ID NO: 2 - DNA, 46 bp, Homo sapiens
gaggaaaatcgggaagcagcttataatgccattactttacctgaag

SEQ ID NO: 3 - DNA, 47 bp, Homo sapiens
tgattttggaatggatgatcgtgagataatgagagaaggcagtgctt

SEQ ID NO: 4 - DNA, 50 bp, Homo sapiens
tgagtttgactcatcagatgaagagcctattgaagatgaacagactccaa

SEQ ID NO: 5 - DNA, 46 bp, Homo sapiens
tcctgacatagccagctgctgtgaaataatggaagagcttacaacc

SEQ ID NO: 6 - DNA, 47 bp, Homo sapiens
ttcgggacaaattagctgcacatctatcatcaagagattcacaatca

SEQ ID NO: 7 - DNA, 44 bp, Homo sapiens
tgcagctggttggtgtcactgccatgtttattgcaagcaaatat

SEQ ID NO: 8 - DNA, 48 bp, Homo sapiens
aacaagtatgccacatcgaagcatgctaagatcagcactctaccacag

SEQ ID NO: 9 - DNA, 47 bp, Homo sapiens
tttagccaaggctgtggcaaaggtgtaacttgtaaacttgagttgga

SEQ ID NO: 10 - DNA, 47 bp, Homo sapiens
catggtgcaaaaataccagtccccagtgagagtgtacaaatacccct

SEQ ID NO: 11 - DNA, 44 bp, Homo sapiens
tcctttgattccgatgttcgtgggcagtgacactgtgagtgaat

SEQ ID NO: 12 - DNA, 48 bp, Homo sapiens
caccctgaaaatgaagattggacctgttttgaacagtctgcaagttta

SEQ ID NO: 13 - DNA, 48 bp, Homo sapiens
catgattgagcaagtgcatgactgtgaaatcattcatggagacattaa

SEQ ID NO: 14 - DNA, 47 bp, Homo sapiens
cttggaaacggatttttggaacaggatgatgaagatgatttatctgc

SEQ ID NO: 15 - DNA, 46 bp, Homo sapiens
tgagatgctcagcaacaaaccatggaactaccagatcgattacttt

SEQ ID NO: 16 - DNA, 47 bp, Homo sapiens
cagactccctcatcaccaaaaagcaagtgtcagtctggtgcagtaat

SEQ ID NO: 17 - DNA, 45 bp, Homo sapiens
caggcctttctgcagaaagcaggcaaatctctgttgttctatgcc

SEQ ID NO: 18 - DNA, 46 bp, Homo sapiens
ttccaggacatcatgcaaaagcaaagaccagaaagagtgtctcatc

SEQ ID NO: 19 - DNA, 48 bp, Homo sapiens
aatgccatcattgctgaacttttgagactctctgagtttattcctgct

SEQ ID NO: 20 - DNA, 48 bp, Homo sapiens
tgggaaagcaaactggatgctaagccagagctacaggatttagatgaa

SEQ ID NO: 21 - DNA, 45 bp, Homo sapiens
caaccaggtgccaaaagaccatccaactatcccgagagctatttc

SEQ ID NO: 22 - DNA, 47 bp, Homo sapiens
tttggttcccttgtgttgattcatactctgaattgtgtacatggaaa

SEQ ID NO: 23 - DNA, 46 bp, Homo sapiens
tttcccacagttgcaaacttgaatagaatcaagttgaacagcaaac

SEQ ID NO: 24 - DNA, 47 bp, Homo sapiens
ggcagagagaggtgctcatgttttctcttgtgggtatcaaaattcta

SEQ ID NO: 25 - DNA, 45 bp, Homo sapiens
ccatccctcgaactcaagtcccgctcattacaaattcttcttgcc

SEQ ID NO: 26 - DNA, 44 bp, Homo sapiens
aagagaggctgcaggaacagcggagaacagttcaggacaagaag

SEQ ID NO: 27 - DNA, 44 bp, Homo sapiens
ccaaaccagccagtcccaagaagaacattaaaactaggagtgcc

SEQ ID NO: 28 - DNA, 42 bp, Homo sapiens
caacaaggccctgagcgtgggtaacatcgatgatgccttaca

SEQ ID NO: 29 - DNA, 47 bp, Homo sapiens
tcatgaaccctttcaacatgcctaatctgtatcagaagttggagagt

SEQ ID NO: 30 - DNA, 47 bp, Homo sapiens
aaaaagagctggggaacgatgcctacaagaagaaagactttgacaca

SEQ ID NO: 31 - DNA, 47 bp, Homo sapiens
cctggacacccaactacaagcagtgttcatggagttcattgaattat

SEQ ID NO: 32 - DNA, 45 bp, Homo sapiens
agaaatgcatgctgtcagcgttggtatttcacattcaatggagct

SEQ ID NO: 33 - DNA, 47 bp, Homo sapiens
accaaggaagccctgaaatgaattcaacaattaatattcatcgcact

SEQ ID NO: 34 - DNA, 47 bp, Homo sapiens
cagtcctgttcagaatgagcaaggctttgtggagttcaaaatttctg

SEQ ID NO: 35 - DNA, 46 bp, Homo sapiens
caatagcaacaggtgcagcagcaagactagtgtcaggatacgacag

SEQ ID NO: 36 - DNA, 46 bp, Homo sapiens
gatccatgcaacctggacttgataaaccggaagattaagtctgtag

SEQ ID NO: 37 - DNA, 45 bp, Homo sapiens
cagcctccacattcagaggcatcacaagtaatggcacaattcttc

SEQ ID NO: 38 - DNA, 46 bp, Homo sapiens
ttctgaaacaagggcgtggatccctcaaccaagaagaatgtttatg

SEQ ID NO: 39 - DNA, 47 bp, Homo sapiens
tgcttggggactattggagaaaataaggtggagtcctacttgtttaa

SEQ ID NO: 40 - DNA, 47 bp, Homo sapiens
agtgcctatggaacatccagctgataatcttgcctagtaagagcaaa

SEQ ID NO: 41 - DNA, 46 bp, Homo sapiens
ttctggcaccatttcgtagccattctctttgtattttaaaaggacg

SEQ ID NO: 42 - DNA, 47 bp, Homo sapiens
cctcaaagaaaccatggccagtagctaggtgttcagtaggaatcaaa

SEQ ID NO: 43 - DNA, 47 bp, Homo sapiens
ttgcacacctgttagcaagaaacagaagttgaaggactggaacaagt

SEQ ID NO: 44 - DNA, 47 bp, Homo sapiens
tcctgtgaaatctccgaggagaagaaagaatgatggacagtttatcc

SEQ ID NO: 45 - DNA, 48 bp, Homo sapiens
gcagcattaagaggtcttctgggagccttaacaagtaccccatattct

SEQ ID NO: 46 - DNA, 48 bp, Homo sapiens
gaattcggaacagatctaacccaaaagtactttctgagaagcagaatg

SEQ ID NO: 47 - DNA, 44 bp, Homo sapiens
aggggtctcatgtggtcctcctcgctatgttggaaatgtgcaac

(19) United States
(12) Patent Application Publication
(10) Pub. No.: US 2009/0298082 A1
(43) Pub. Date: Dec. 3, 2009

(54) BIOMARKER PANELS FOR PREDICTING PROSTATE CANCER OUTCOMES

(76) Inventors: George G. Klee, Rochester, MN (US); Robert B. Jenkins, Rochester, MN (US); Thomas M. Kollmeyer, Rochester, MN (US); Karla V. Ballman, Northfield, MN (US); Eric J. Bergstralh, Mazeppa, MN (US); Bruce W. Morlan, Northfield, MN (US); S. Keith Anderson, Rochester, MN (US); Tohru Nakagawa, Tokyo (JP)

Correspondence Address: FISH & RICHARDSON P.C., PO BOX 1022, MINNEAPOLIS, MN, 55440-1022, US

(21) Appl. No.: 12/474,879

(22) Filed: May 29, 2009

Related U.S. Application Data
(60) Provisional application No. 61/057,698, filed on May 30, 2008.

(51) Int. Cl. C12Q 1/68

(52) U.S. Cl. 435/6

(57) ABSTRACT

This document provides methods and materials related to assessing male mammals (e.g., humans) with prostate cancer. For example, methods and materials for predicting (1) which patients, at the time of PSA reoccurrence, will later develop systemic disease, (2) which patients, at the time of retropubic radial prostatectomy, will later develop systemic disease, and (3) which patients, at the time of systemic disease, will later die from prostate cancer are provided.

### CROSS-REFERENCE TO RELATED APPLICATIONS

This application claims the benefit of priority to U.S. Provisional Application Ser. No. 61/057,698, filed May 30, 2008. The disclosure of the prior application is considered part of (and is incorporated by reference in) the disclosure of this application.

### STATEMENT AS TO FEDERALLY SPONSORED RESEARCH

Funding for the work described herein was provided by the federal government under grant number 90966043 awarded by the National Institute of Health. The federal government has certain rights in the invention.

### BACKGROUND

1. Technical Field

This document relates to methods and materials involved in predicting the outcome of prostate cancer.

2. Background Information

Prostate cancer occurs when a malignant tumor forms in the tissue of the prostate. The prostate is a gland in the male reproductive system located below the bladder and in front of the rectum. The main function of the prostate gland, which is about the size of a walnut, is to make fluid for semen. Although there are several cell types in the prostate, nearly all prostate cancers start in the gland cells. This type of cancer is known as adenocarcinoma.

Prostate cancer is the second leading cause of cancer-related death in American men. Most of the time, prostate cancer grows slowly. Autopsy studies show that many older men who died of other diseases also had prostate cancer that neither they nor their doctor were aware of. Sometimes, however, prostate cancer can grow and spread quickly. It is important to be able to distinguish prostate cancers that will grow slowly from those that will grow quickly since treatment can be especially effective when the cancer has not spread beyond the region of the prostate. Finding ways to detect cancers early can improve survival rates.

### SUMMARY

This document provides methods and materials related to assessing male mammals (e.g., humans) with prostate cancer. For example, this document provides methods and materials for predicting (1) which patients, at the time of PSA reoccurrence, will later develop systemic disease, (2) which patients, at the time of retropubic radial prostatectomy, will later develop systemic disease, and (3) which patients, at the time of systemic disease, will later die from prostate cancer.

The majority of men with prostate cancer are diagnosed with cancers with low mortality. Initial treatment is typically radical prostatectomy, external beam radiotherapy, or brachytherapy and followed by serial serum PSA measurements. Not every man who suffers PSA recurrence is destined to suffer systemic progression or to die of his prostate cancer. Thus, it is not clear whether men with PSA recurrence should be simply observed or should receive early androgen ablation. The methods and materials provided herein can be used to predict which men with a rising PSA post-definitive therapy might benefit from additional therapy.

In general, one aspect of this document features a method for predicting whether or not a human, at the time of PSA reoccurrence or retropubic radial prostatectomy, will later develop systemic disease. The method comprises, or consists essentially of, (a) determining an expression profile score for cancer tissue from the human, wherein the expression profile score is based on at least the expression levels of RAD21, CDKN3, CCNB1, SEC14L1, BUB1, ALAS1, KIAA0196, TAF2, SFRP4, STIP1, CTHRC1, SLC44A1, IGFBP3, EDG7, FAM49B, C8orf53, and CDK10 nucleic acid, and

(b) prognosing the human as later developing systemic disease or as not later developing systemic disease based on at least the expression profile score. The method can be performed at the time of the PSA reoccurrence. The method can be performed at the time of the retropubic radial prostatectomy. The expression levels can be mRNA expression levels. The prognosing step (b) can comprise prognosing the human as later developing systemic disease or as not later developing systemic disease based on at least the expression profile score and a clinical variable. The clinical variable can be selected from the group consisting of a Gleason score and a revised Gleason score. The clinical variable can be selected from the group consisting of a Gleason score, a revised Gleason score, the pStage, age at surgery, initial PSA at recurrence, use of hormone or radiation therapy after radical retropubic prostatectomy, age at PSA recurrence, the second PSA level at time of PSA recurrence, and PSA slope. The method can comprise prognosing the human as later developing systemic disease based on at least the expression profile score. The method can comprise prognosing the human as not later developing systemic disease based on at least the expression profile score.

In another aspect, this document features a method for predicting whether or not a human, at the time of systemic disease, will later die from prostate cancer. The method comprises, or consists essentially of, (a) determining an expression profile score for cancer tissue from the human, wherein the expression profile score is based on at least the expression levels of RAD21, CDKN3, CCNB1, SEC14L1, BUB1, ALAS1, KIAA0196, TAF2, SFRP4, STIP1, CTHRC1, SLC44A1, IGFBP3, EDG7, FAM49B, C8orf53, and CDK10 nucleic acid, and (b) prognosing the human as later dying of the prostate cancer or as not later dying of the prostate cancer based on at least the expression profile score. The expression levels can be mRNA expression levels. The prognosing step (b) can comprise prognosing the human as later developing systemic disease or as not later developing systemic disease based on at least the expression profile score and a clinical variable. The clinical variable can be selected from the group consisting of a Gleason score and a revised Gleason score. The clinical variable can be selected from the group consisting of a Gleason score, a revised Gleason score, the pStage, age at surgery, initial PSA at recurrence, use of hormone or radiation therapy after radical retropubic prostatectomy, age at PSA recurrence, the second PSA level at time of PSA recurrence, and PSA slope. The method can comprise prognosing the human as later dying of the prostate cancer based on at least the expression profile score. The method can comprise prognosing the human as not later dying of the prostate cancer based on at least the expression profile score.

In another aspect, this document features a method for (1) predicting whether or not a patient, at the time of PSA reoccurrence, will later develop systemic disease, (2) predicting whether or not a patient, at the time of retropubic radial prostatectomy, will later develop systemic disease, or (3) predicting whether or not a patient, at the time of systemic disease, will later die from prostate cancer. The method comprises, or consists essentially of, determining whether or not cancer tissue from the patient contains an RAD21, CDKN3, CCNB1, SEC14L1, BUB1, ALAS1, KIAA0196, TAF2, SFRP4, STIP1, CTHRC1, SLC44A1, IGFBP3, EDG7, FAM49B, C8orf53, and CDK10 expression profile indicative of a later development of the systemic disease or the death.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1: Nine genes with significantly different expression in cases with systemic disease progression (SYS) versus controls with PSA recurrence (PSA). P-values (t-test) for the SYS case/PSA control comparison are shown. Controls with no evidence of disease recurrence (NED) are also included.

FIG. 2: (A to D) Areas under the curve (AUCs) for three clinical models, the final 17 gene/probe model and the combined clinical probe models. A. The training set AUCs for three clinical models, the final 17 gene/probe model and the combined clinical/17 gene/probe model. B. The validation set AUCs for three clinical models, the final 17 gene/probe model and the combined clinical/17 gene/probe model. C. The training set AUCs of four previously reported gene expression models of prostate cancer aggressiveness compared with the Clinical model C alone and with the 17 gene/probe model. D. The validation set AUCs of four previously reported gene expression models of prostate cancer aggressiveness compared with the clinical model C alone and with the 17 gene/probe model. For an explanation of the clinical models see Table 4. (E and F) A comparison of the training and validation set AUCs for each of the model. E. AUCs of the each of the gene/probe models alone. F. AUCs of each of the gene/probe models with the inclusion of clinical model C.

FIG. 3: Systemic progression-free and overall prostate cancer-specific survival in the PSA Control and SYS Case groups. A) Systemic progression-free survival for the patients classified in the poor outcome category and for those in the good outcome category in the PSA control group—17 gene/probe model. B) Prostate cancer-specific overall survival for the patients classified in the poor outcome category and for those in the good outcome category in the SYS case group—17 gene/probe model. C) Prostate cancer-specific overall survival for patients classified in the poor outcome category and for those in the good outcome category in the SYS case group—Lapointe et al. 2004 recurrence model.

FIG. 4: Expression results for ERG, ETV1 and ETV4 among the men with no evidence of disease progression (NED), PSA recurrence (PSA) and systemic progression (SYS). (A) Each overlapping set of three bars represent a different a different case or control. Thresholds for overexpression are ERG >3200, ETV1 >6000 and ETV4 >1400. (B) The numbers of cases showing overexpression of one or more of ERG, ETV1 and ETV4 are shown.

FIG. 5 is a summary of the nested case-control study design.

FIG. 6: Reproducibility of DASL assay and the effect of RNA quantity on the DASL assay. (A) An example of DASL interplate reproducibility. (B) Effect of reduced RNA quantity on the DASL assay.

FIG. 7: (A to E) Example results of the comparison of quantitative RT-PCR and DASL data on ERG—Cancer Panel ver1 (A, R2=0.94), ERG—Custom Panel (B, R2=0.94), PAGE4 (C, R2=0.89), MUC1 (D, R2=0.82), and FAM13C1 (E, R2=0.75). (F) Summary of quantitative RT-PCR and DASL data comparisons.

FIG. 8: Comparison of genes having multiple probe sets on the Cancer Panel v1 and/or the Custom panel. (A) Comparison of three probe sets (Cancer Panel ERG, Custom Panel ERG and Custom panel ERG splice variant) for ERG. (B) Comparison of two probe sets (Custom Panel SRD5A2 and Custom panel terparbo) for SRD5A2/terparbo.

### DETAILED DESCRIPTION

This document provides methods and materials related to assessing male mammals (e.g., humans) with prostate cancer. For example, this document provides methods and materials for predicting (1) which patients, at the time of PSA reoccurrence, will later develop systemic disease, (2) which patients, at the time of retropubic radial prostatectomy, will later develop systemic disease, and (3) which patients, at the time of systemic disease, will later die from prostate cancer. As described herein, the expression level of any of the genes listed in the tables provided herein (e.g., Tables 2 and 3) or any combination of the genes listed in the tables provided herein can be assessed as described herein to predict (1) which patients, at the time of PSA reoccurrence, will later develop systemic disease, (2) which patients, at the time of retropubic radial prostatectomy, will later develop systemic disease, and (3) which patients, at the time of systemic disease, will later die from prostate cancer. For example, the combination of genes set forth in Table 3 can be assessed as described herein to predict (1) which patients, at the time of PSA reoccurrence, will later develop systemic disease, (2) which patients, at the time of retropubic radial prostatectomy, will later develop systemic disease, and (3) which patients, at the time of systemic disease, will later die from prostate cancer.

Any appropriate type of sample (e.g., cancer tissue) can be used to assess the level of gene expression. For example, prostate cancer tissue can be collected and assessed to determine the expression level of a gene listed in any of the tables provided herein. Once obtained, the expression level for a particular nucleic acid can be used as a raw number or can be normalized using appropriate calculations and controls. In addition, the expression levels for groups of nucleic acids can be combined to obtain an expression level score that is based on the measured expression levels (e.g., raw expression level number or normalized number). In some cases, the expression levels of the individual nucleic acids that are used to obtain an expression level score can be weighted. An expression level score can be a whole number, an integer, an alphanumerical value, or any other representation capable of indicating whether or not a condition is met. In some cases, an expression level score is a number that is based on the mRNA expression levels of at least the seventeen nucleic acids listed in Table 3. In some cases, an expression level score can be based on the mRNA expression levels of the seventeen nucleic acids listed in Table 3 and no other nucleic acids. As described herein, the seventeen nucleic acids listed in Table 3 can be used together to determine, at the time of PSA reoccurrence or at the time of retropubic radial prostatectomy, whether or not a mammal will later develop systemic disease. In addition, the seventeen nucleic acids listed in Table 3 can be used together to determine, at the time of systemic disease, whether or not a mammal will later die of prostate cancer.

For humans, the seventeen nucleic acids listed in Table 3 can have the nucleic acid sequence set forth in GenBank as follows: RAD21 (GenBank Accession No. NM_{—}006265; GI No. 208879448; probe sequences GGGATAAGAAGCTAACCAAAGCCCATGTGTTCGAGTGTAATTTAGAGAG (SEQ ID NO:1), GAGGAAAATCGGGAAGCAGCTTATAATGCCATTACTTTACCTGAAG (SEQ ID NO:2), and TGATTTTGGAATGGATGATCGTGAGATAATGAGAGAAGGCAGTGCTT (SEQ ID NO:3)), CDKN3 (GenBank Accession Nos. NM_{—}005192 and NM_{—}001130851; GI Nos. 195927023 and 195927024; probe sequences TGAGTTTGACTCATCAGATGAAGAGCCTATTGAAGATGAACAGACTCCAA (SEQ ID NO:4), TCCTGACATAGCCAGCTGCTGTGAAATAATGGAAGAGCTTACAACC (SEQ ID NO:5), and TTCGGGACAAATTAGCTGCACATCTATCATCAAGAGATTCACAATCA (SEQ ID NO:6)), CCNB1 (GenBank Accession No. NM_{—}031966; GI No. 34304372; probe sequences TGCAGCTGGTTGGTGTCACTGCCATGTTTATTGCAAGCAAATAT (SEQ ID NO:7), AACAAGTATGCCACATCGAAGCATGCTAAGATCAGCACTCTACCACAG (SEQ ID NO:8), and TTTAGCCAAGGCTGTGGCAAAGGTGTAACTTGTAAACTTGAGTTGGA (SEQ ID NO:9)), SEC14L1 (GenBank Accession Nos. NM_{—}001039573, NM_{—}001143998, NM_{—}001143999, NM_{—}001144001, and NM_{—}003003; GI Nos. 221316683, 221316675, 221316679, 221316686, and 221316681; probe sequences CATGGTGCAAAAATACCAGTCCCCAGTGAGAGTGTACAAATACCCCT (SEQ ID NO:10), TCCTTTGATTCCGATGTTCGTGGGCAGTGACACTGTGAGTGAAT (SEQ ID NO: 11), and CACCCTGAAAATGAAGATTGGACCTGTTTTGAACAGTCTGCAAGTTTA (SEQ ID NO:12)), BUB1 (GenBank Accession No. NM_{—}004336; GI No. 211938448; probe sequences CATGATTGAGCAAGTGCATGACTGTGAAATCATTCATGGAGACATTAA (SEQ ID NO:13), CTTGGAAACGGATTTTTGGAACAGGATGATGAAGATGATTTATCTGC (SEQ ID NO:14), and TGAGATGCTCAGCAACAAACCATGGAACTACCAGATCGATTACTTT (SEQ ID NO:15)), ALAS1 (GenBank Accession Nos. NM_{—}000688 and NM_{—}199166; GI Nos. 40316942 and 40316938; probe sequences CAGACTCCCTCATCACCAAAAAGCAAGTGTCAGTCTGGTGCAGTAAT (SEQ ID NO:16), CAGGCCTTTCTGCAGAAAGCAGGCAAATCTCTGTTGTTCTATGCC (SEQ ID NO: 17), and TTCCAGGACATCATGCAAAAGCAAAGACCAGAAAGAGTGTCTCATC (SEQ ID NO:18)), KIAA0196 (GenBank Accession No. NM_{—}014846; GI No. 120952850; probe sequences AATGCCATCATTGCTGAACTTTTGAGACTCTCTGAGTTTATTCCTGCT (SEQ ID NO:19), TGGGAAAGCAAACTGGATGCTAAGCCAGAGCTACAGGATTTAGATGAA (SEQ ID NO:20), and CAACCAGGTGCCAAAAGACCATCCAACTATCCCGAGAGCTATTTC (SEQ ID NO:21)), TAF2 (GenBank Accession No. NM_{—}003184; GI No. 115527086; probe sequences TTTGGTTCCCTTGTGTTGATTCATACTCTGAATTGTGTACATGGAAA (SEQ ID NO:22), TTTCCCACAGTTGCAAACTTGAATAGAATCAAGTTGAACAGCAAAC (SEQ ID NO:23), and GGCAGAGAGAGGTGCTCATGTTTTCTCTTGTGGGTATCAAAATTCTA (SEQ ID NO:24)), SFRP4 (GenBank Accession No. NM_{—}003014; GI No. 170784837; probe sequences CCATCCCTCGAACTCAAGTCCCGCTCATTACAAATTCTTCTTGCC (SEQ ID NO:25), AAGAGAGGCTGCAGGAACAGCGGAGAACAGTTCAGGACAAGAAG (SEQ ID NO:26), and CCAAACCAGCCAGTCCCAAGAAGAACATTAAAACTAGGAGTGCC (SEQ ID NO:27)), STIP1 (GenBank Accession No. NM_{—}006819; GI No. 110225356; probe sequences CAACAAGGCCCTGAGCGTGGGTAACATCGATGATGCCTTACA (SEQ ID NO:28), TCATGAACCCTTTCAACATGCCTAATCTGTATCAGAAGTTGGAGAGT (SEQ ID NO:29), and AAAAAGAGCTGGGGAACGATGCCTACAAGAAGAAAGACTTTGACACA (SEQ ID NO:30)), CTHRC1 (GenBank Accession No. NM_{—}138455; GI No. 34147546; probe sequences CCTGGACACCCAACTACAAGCAGTGTTCATGGAGTTCATTGAATTAT (SEQ ID NO:31), AGAAATGCATGCTGTCAGCGTTGGTATTTCACATTCAATGGAGCT (SEQ ID NO:32), ACCAAGGAAGCCCTGAAATGAATTCAACAATTAATATTCATCGCACT (SEQ ID NO:33)), SLC44A1 (GenBank Accession No. NM_{—}080546; GI No. 112363101; probe sequences CAGTCCTGTTCAGAATGAGCAAGGCTTTGTGGAGTTCAAAATTTCTG (SEQ ID NO:34), CAATAGCAACAGGTGCAGCAGCAAGACTAGTGTCAGGATACGACAG (SEQ ID NO:35), and GATCCATGCAACCTGGACTTGATAAACCGGAAGATTAAGTCTGTAG (SEQ ID NO:36)), IGFBP3 (GenBank Accession Nos. NM_{—}000598 and NM_{—}001013398; GI Nos. 62243067 and 62243247; probe sequences CAGCCTCCACATTCAGAGGCATCACAAGTAATGGCACAATTCTTC (SEQ ID NO:37), TTCTGAAACAAGGGCGTGGATCCCTCAACCAAGAAGAATGTTTATG (SEQ ID NO:38), and TGCTTGGGGACTATTGGAGAAAATAAGGTGGAGTCCTACTTGTTTAA (SEQ ID NO:39)), EDG7 (GenBank Accession No. NM_{—}012152; GI No. 183396778; probe sequences AGTGCCTATGGAACATCCAGCTGATAATCTTGCCTAGTAAGAGCAAA (SEQ ID NO:40), TTCTGGCACCATTTCGTAGCCATTCTCTTTGTATTTTAAAAGGACG (SEQ ID NO:41), and CCTCAAAGAAACCATGGCCAGTAGCTAGGTGTTCAGTAGGAATCAAA (SEQ ID NO:42)), FAM49B (GenBank Accession No. NM_{—}016623; GI No. 42734437; probe sequences TTGCACACCTGTTAGCAAGAAACAGAAGTTGAAGGACTGGAACAAGT (SEQ ID NO:43), TCCTGTGAAATCTCCGAGGAGAAGAAAGAATGATGGACAGTTTATCC (SEQ ID NO:44), and GCAGCATTAAGAGGTCTTCTGGGAGCCTTAACAAGTACCCCATATTCT (SEQ ID NO:45)), C8orf53 (GenBank Accession No. NM_{—}032334; GI No. 223468686; probe sequence GAATTCGGAACAGATCTAACCCAAAAGTACTTTCTGAGAAGCAGAATG (SEQ ID NO:46)), and CDK10 (GenBank Accession Nos. NM_{—}001098533, NM_{—}001160367, NM_{—}052987, and NM_{—}052988; GI Nos. 237858579, 237858581, 237858574, and 237858573; probe sequence AGGGGTCTCATGTGGTCCTCCTCGCTATGTTGGAAATGTGCAAC (SEQ ID NO:47)).

Any appropriate method can be used to determine the expression level of a gene listed herein. For example, reverse transcription-PCR (RT-PCR) techniques can be performed to detect the level of gene expression.

The term “elevated level” as used herein with respect to the level of mRNA for a nucleic acid listed herein is any mRNA level that is greater than a reference mRNA level for that nucleic acid. The term “reference level” as used herein with respect to an mRNA for a nucleic acid listed herein is the level of mRNA for a nucleic acid listed herein that is typically expressed by mammals with prostate cancer that does not progress to systemic disease or result in prostate cancer-specific death. For example, a reference level of an mRNA biomarker listed herein can be the average mRNA level of that biomarker that is present in samples obtained from a random sampling of 50 males without prostate cancer.

It will be appreciated that levels from comparable samples are used when determining whether or not a particular level is an elevated level. For example, the average mRNA level present in bulk prostate tissue from a random sampling of mammals may be X units/g of prostate tissue, while the average mRNA level present in isolated prostate epithelial cells may be Y units/number of prostate cells. In this case, the reference level in bulk prostate tissue would be X units/g of prostate tissue, and the reference level in isolated prostate epithelial cells would be Y units/number of prostate cells. Thus, when determining whether or not the level in bulk prostate tissue is elevated, the measured level would be compared to the reference level in bulk prostate tissue. In some cases, the reference level can be a ratio of an expression value of a biomarker in a sample to an expression value of a control nucleic acid or polypeptide in the sample. A control nucleic acid or polypeptide can be any polypeptide or nucleic acid that has a minimal variation in expression level across various samples of the type for which the nucleic acid or polypeptide serves as a control. For example, GAPDH, HPRT, NDUFA7, and RPS16 nucleic acids or polypeptides can be used as control nucleic acids or polypeptides, respectively, in prostate samples. In some cases, nucleic acids or polypeptides can be used as control nucleic acids or polypeptides, respectively, as described elsewhere (Ohl et al., J. Mol. Med., 83:1014-1024 (2005)).

Once determined, the level of mRNA expression for a particular nucleic acid listed herein (or the degree of which the level is elevated over a reference level) can be combined with the levels of mRNA expression for other particular nucleic acids listed herein to obtain an expression level score. For example, the mRNA levels for each nucleic acid listed in Table 3 can be added together to obtain an expression level score. If this expression level score is greater than the sum of corresponding mRNA reference levels for each nucleic acid listed in Table 3, then the patient, at the time of PSA reoccurrence or retropubic radial prostatectomy, can be classified as later developing systemic disease or, at the time of systemic disease, can be classified as later dying from prostate cancer.

In some cases, the levels of biomarkers (e.g., an expression level score) can be used in combination with one or more other factors to assess a prostate cancer patient. For example, expression level scores can be used in combination with the clinical stage, the serum PSA level, and/or the Gleason score of the prostate cancer to determine, at the time of PSA reoccurrence or at the time of retropubic radial prostatectomy, whether or not a mammal will later develop systemic disease. In addition, such combinations can be used together to determine, at the time of systemic disease, whether or not a mammal will later die of prostate cancer. Additional information about the mammal, such as information concerning genetic predisposition to develop cancer, SNPs, chromosomal abnormalities, gene amplifications or deletions, and/or post translational modifications, can also be used in combination with the level of one or more biomarkers provided herein (e.g., the list of nucleic acids set forth in Table 3) to assess prostate cancer patients.

This document also provides methods and materials to assist medical or research professionals in determining, at the time of PSA reoccurrence or at the time of retropubic radial prostatectomy, whether or not a mammal will later develop systemic disease or in determining, at the time of systemic disease, whether or not a mammal will later die of prostate cancer. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the level of one or more than one biomarker in a sample, and (2) communicating information about that level to that professional.

Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

The invention will be further described in the following examples, which do not limit the scope of the invention described in the claims.

### EXAMPLES

### Example 1

### A Tissue Biomarker Panel that Predicts which Men with a Rising PSA Post-Definitive Prostate Cancer Therapy Will Have Systemic Progression

After therapy for prostate cancer many men develop a rising PSA. Such men may develop a local or metastatic recurrence that warrants further therapy. However many men will have no evidence of disease progression other than the rising PSA and will have a good outcome. A case-control design, incorporating test and validation cohorts, was used to test the association of gene expression results with outcome after PSA progression. Using arrays optimized for paraffin-embedded tissue RNAs, a gene expression model significantly associated with systemic progression after PSA progression was developed. The model also predicted prostate cancer death (in men with systemic progression) and systemic progression beyond 5 years (in PSA controls) with hazard ratios 2.5 and 4.7, respectively (log-rank p-values of 0.0007 and 0.0005). The measurement of gene expression pattern may be useful for determining which men may benefit from additional therapy after PSA recurrence.

### Gene Selection and Array Design for the DASL™ Assay:

Two Illumina DASL expression microarrays were utilized for the experiments: (1) The standard commercially available Illumina DASL expression microarray (Cancer Panel™ v1) containing 502 oncogenes, tumor suppressor genes and genes in their associated pathways. Seventy-eight of the targets on the commercial array have been associated with prostate cancer progression. (2) A custom Illumina DASL™ expression microarray containing 526 gene targets for RNAs, including genes whose expression is altered in association with prostate cancer progression. Four different sets of prostate cancer aggressiveness genes were included in the study. If the genes were not present on the Cancer Panel v1 array, then they were included in the design of the custom array:

1) Markers of prostate cancer aggressiveness identified by a Mayo/University of Minnesota Partnership (Kube et al., BMC Mol. Biol., 8:25 (2007)): The expression profiles of 100 laser-capture microdissected prostate cancer lesions and matched normal and BPH control lesions were analyzed using Affymetrix HG-U133 Plus 2.0 microarrays. Ranked lists of significantly over- and under-expressed genes comparing 10 Gleason 5 and 7 metastatic lesions to 31 Gleason 3 cancer lesions were generated. The top 500 genes on this list were compared to lists generated from prior expression microarray studies and other marker studies of prostate cancer (see 2-4 next). After this analysis there was space for 204 novel targets with potential association with aggressive prostate cancer on the custom array.

2) Markers associated with prostate cancer aggressiveness from publicly available expression microarray datasets (e.g. EZH2, AMACR, hepsin, PRLz, PRL3): Sufficiently large datasets from 9 prior microarray studies of prostate cancer of varying grades and metastatic potential (Dhanasekaran et al., Nature. 412, 822-826 (2001); Luo et al., Cancer Res. 61, 4683-4688 (2001); Magee et al., Cancer Res. 61, 5692-5696 (2001); Welsh et al., Cancer Res. 61, 5974-5978 (2001); LaTulippe et al., Cancer Res. 62, 4499-4506 (2002), Singh et al., Cancer Cell. 1, 203-209 (2002); Glinsky et al., J Clin Invest. 113, 913-923 (2004); Lapointe et al., Proc Natl Acad Sci USA. 101, 811-816 (2004); and Yu et al., J Clin Oncol. 22, 2790-2799 (2004)) were available from the OncoMine internet site (Rhodes et al., Neoplasia. 6, 1-6 (2004); Rhodes et al., Proc Natl Acad Sci USA. 101, 9309-9314 (2004); www.oncomine.org) when the array was designed. From ordered lists of these data, 32 genes were selected for inclusion on the array.

3) Previously published markers associated with prostate cancer aggressiveness (e.g. PSMA, PSCA, Cav-1): Expression microarray data has also been published. This literature was evaluated for additional tissue biomarkers. For example, at the time of array design 13 high quality expression microarray studies of prostate cancer aggressiveness were identified (See Supplemental Tables 1 and 2 of U.S. Provisional Patent Application No. 61/057,698, filed May 30, 2008, for full reference list). In addition, among the 13 reports, 5 papers presented 8 expression biomarker panels to predict prostate cancer aggressiveness (Singh et al., Cancer Cell. 1, 203-209 (2002); Glinsky et al., J Clin Invest. 113, 913-923 (2004); Lapointe et al., Proc Natl Acad Sci USA. 101, 811-816 (2004); Yu et al., J Clin Oncol. 22, 2790-2799 (2004); and Glinsky et al., J Clin Invest. 115, 1503-1521 (2005)). When appropriate probes suitable for the DASL chemistry could be designed for these panels they were included on the custom array. 12 articles were identified reviewing genes associated with prostate cancer. These criteria resulted in the selection of 150 genes.

4) Markers derived from Mayo SPORE research (including genes and ESTs mapped to 8q24). Ninety-three additional biomarkers were identified (see Supplemental Tables 1 and 2 of U.S. Provisional Patent Application No. 61/057,698, filed May 30, 2008).

The custom array also included probe sets for 47 genes that were not expected to differ between case and control groups. Thirty-eight of these genes were also present on the commercial array (see Supplemental Tables 1 and 2 of U.S. Provisional Patent Application No. 61/057,698, filed May 30, 2008).

After enumerating the potentially prostate cancer relevant genes on the commercially available cancer panel, 557 potentially prostate cancer relevant genes and 424 other cancer-related genes were evaluated across both arrays.

### Design of Nested Case-Control Study:

Since training and validation analysis requires tissue from patients with sufficient follow-up time, for this study individuals from the Mayo Radical Retropubic Prostatectomy (RRP) Registry were sampled. The registry consists of a population of men who received prostatectomy as their first treatment for prostate cancer at the Mayo Clinic (For a current description and use of the registry; see Tollefson et al., Mayo Clin Proc. 82, 422-427 (2007)). As systemic progression is relatively infrequent, a case-control study nested within a cohort of men with a rising PSA was designed. Between 1987-2001, inclusive, 9,989 previously-untreated men had RRP at Mayo. On follow-up, 2,131 developed a rising PSA (>30 days after RRP) in the absence of concurrent clinical recurrence. PSA rise was defined as a follow-up PSA>=0.20 ng/ml, with the next PSA at least 0.05 ng/ml higher or the initiation of treatment for PSA recurrence (for patients whose follow-up PSA was high enough to warrant treatment). This group of 2,131 men comprises the underlying cohort from which SYS cases and PSA controls were selected.

Within 5 years of PSA rise, 213 men developed systemic progression (SYS cases), defined as a positive bone scan or CT scan. Of these, 100 men succumbed to a prostate cancer-specific death, 37 died from other causes, and 76 remain at risk.

PSA progression controls (213) were selected from those men without systemic progression within 5 years after the PSA rise and were matched (1:1) on birth year, calendar year of PSA rise and initial diagnostic pathologic Gleason score (<=6, 7+). Twenty of these men developed systemic progression greater than 5 years after initial PSA rise and 9 succumbed to a prostate cancer-specific death.

A set of 213 No Evidence of Disease (NED) Progression controls were also selected from the Mayo RRP Registry of 9,989 men and used for some comparisons. These controls had RRP from 1987-1998 with no evidence of PSA rise within 7 years of RRP. The median (25th, 75th percentile) follow-up from RRP was 11.3 (9.3, 13.8) years. The NED controls were matched to the systemic progression cases on birth-year, calendar year of RRP and initial diagnostic Gleason Score. Computerized optimal matching was performed to minimize the total “distance” between cases and controls in terms of the sum of the absolute difference in the matching factors (Bergstralh et al., Epidemiology. 6, 271-275 (1995)).

### Block Identification, RNA Isolation, and Expression Analysis:

The list of 639 cases and controls was randomized. An attempt was made to identify all available blocks from the RRP (including apparently normal and abnormal lymph nodes) from the randomized list of 639 eligible cases and controls. Maintaining the randomization, each available block was assessed for tissue content by pathology review, and the block containing the dominant Gleason pattern cancer was selected for RNA isolation.

Four freshly cut 10 μm sections of FFPE tissue were deparaffinized and the Gleason dominant cancer focus was macrodissected. RNA was extracted using the High Pure RNA Paraffin Kit from Roche (Indianapolis, Ind.). RNA was quantified using ND-1000 spectrophotometer from NanoDrop Technologies (Wilmington, Del.). The RNAs were distributed on 96-well plates in the randomized order for DASL analysis (including within-run and between-run duplicates).

Probes for the custom DASL® panel were designed and synthesized by Illumina, Inc. (San Diego, Calif.). RNA samples were processed in following the manufacturer's manual. Samples were hybridized to Sentrix Universal 96-Arrays and scanned using Illumina's BeadArray Reader.

In order to evaluate the accuracy of the gene expression levels defined by the DASL technology, quantitative SYBR Green RT-PCR reactions were performed for 9 selected “target” genes (CDH1, MUC1, VEGF, IGFBP3, ERG, TPD52, YWHAZ, FAM13C1, and PAGE4) and four commonly-used endogenous control genes (GAPDH, B2M, PPIA and RPL13a) in 384-well plates, with the use of Prism 7900HT instruments (Applied Biosystems, Foster City, Calif.). 210 RNA samples with abundant mRNA from the group of total 639 patients were analyzed. For the PAGE4 assay, only 77 samples were subjected to the assay because of mRNA shortage. mRNA was reverse-transcriptized with SuperScript III First Strand Synthesis SuperMix (Invitrogen, Carlsbad, Calif.) for first strand synthesis using random hexamer. Expression of each gene was measured (the number of cycles required to achieve a threshold, or Ct) in triplicate and then normalized relative to the set of four reference genes.

### Pathology Review:

The Gleason score in the Mayo Clinic RRP Registry was the initial diagnostic Gleason score. Since there have been changes in pathologic interpretation of the Gleason Score over time, a single pathologist (JCC) reviewed the Gleason score of each of the blocks selected for expression analysis. This clinical variable was designated as the revised Gleason Score.

### Statistical Methodology:

Collection of gene expression data was attempted for the 623 patients as described herein. Of these, there were 596 (nSYS=200, nPSA=201, nNED=195) patients for whom data was collected, the rest having failed one or both expression panels as described herein. To assure selection of similar training and validation sets, 100 case-control-control cohorts comprised of 133 randomly chosen SYS patients (two-thirds of 200 for training) along with their matched PSA and NED controls were selected as a proposed training set. The remaining cases and controls were treated as a proposed validation set. The clinical variables were tested for independence between the proposed training and validation sets separately within the SYS cases and the PSA controls. Discrete clinical factors (pathologic stage, hormonal treatment adjuvant to RRP, radiation treatment adjuvant to RRP, hormonal treatment adjuvant to PSA recurrence, and radiation therapy adjuvant to PSA recurrence) were tested using Chi-square analysis. Continuous clinical variables (Gleason score (revised), age at PSA recurrence, first rising PSA value, second rising PSA value, and PSA slope) were tested using Wilcoxon rank sum. Six of the one hundred randomly sampled sets failed to show dependency for any of the clinical variables at the 0.2 level, and the first of these was chosen as the training set: 391 patients (nSYS=133, nPSA=133, nNED=125). This reserved 205 patients for the validation set (nSYS=67, nPSA=68, nNED=70).

The purpose of array normalization is to remove systemic biases introduced during the sample preparation, hybridization, and scanning process. Since different samples were randomly assigned to arrays and positions on arrays, the data was normalized by total fluorescence separately within each disease group within each array type. The normalization technique used was fast cyclic loess (fastlo) (Ballman et al., Bioinformatics. 20, 2778-2786 (2004)).

The training data were analyzed using random forests (Breiman, Machine Learning. 45, 5-32 (2001)) using R Version 2.3.1 (http://www.r-project.org) and randomForest version 4.5-16 (http://stat-www.berkeley.edu/users/breiman/RandomForests). The data were analyzed by panel (Cancer, Custom and Merged, where Merged was the Cancer and Custom data treated as a single array). By testing the ntree parameter of the randomForest function, it was determined that 4000 random forests were sufficient to generate a stable list of markers. The top markers as sorted for significance by the randomForest program were combined with various combinations of clinical variables using logistic regression R program (glm( ) with family=binary (a logistic model), where glm refers to generalized linear model). The resulting scoring function was then analyzed using Receiver Operating Characteristic (ROC) methods, and the cut-off was chosen that assumed an equal penalty for false positives and false negatives. A review of the models permitted a subset of markers to be identified, and a subset of supporting clinical data identified. The number of features in the model was determined by leave ⅓ out Monte Carlo Cross Validation (MCCV) using 100 iterations. The number of features was selected to maximize AUC and minimize random variation in the model. The final model was then applied to the 391 patient training set and the reserved 205 patient validation set. For comparison, other previously reported gene expression models were also tested against the training and validation sets (Singh et al., Cancer Cell. 1, 203-209 (2002); Glinsky et al., J Clin Invest. 113, 913-923 (2004); Lapointe et al., Proc Natl Acad Sci USA. 101, 811-816 (2004); Yu et al., J Clin Oncol. 22, 2790-2799 (2004); and Glinsky et al., J Clin Invest. 115, 1503-1521 (2005)).

### Study Design/Paraffin Block Recovery/RNA Isolation and Expression Panel Success

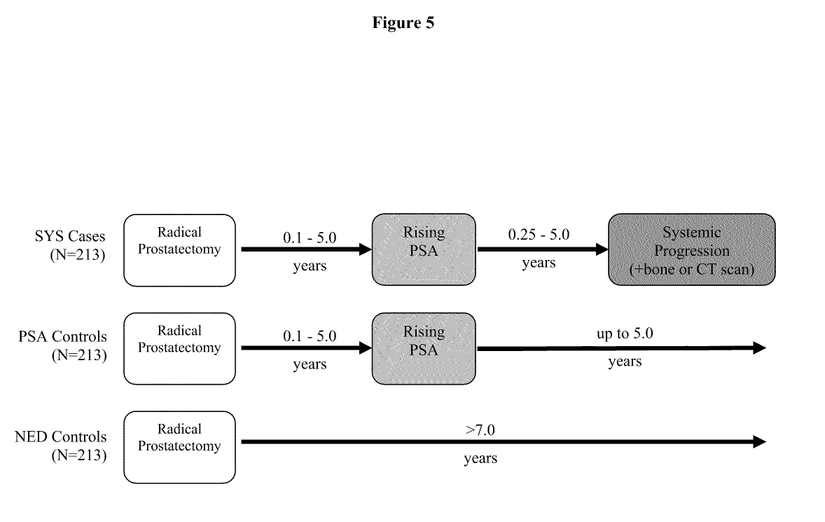

Briefly, a nested case-control study was performed using the large, well-defined cohort of men with rising PSA following radical prostatectomy at our institution. FIG. 5 summarizes the study design. SYS cases were 213 men who developed systemic progression between 90 days and 5.0 years following the PSA rise. PSA control were a random sample of 213 men post-radical prostatectomy with PSA recurrence with no evidence of further clinical progression within 5 years. NED controls were a random sample of 213 men post-radical prostatectomy without PSA rise within 7 years (the comparison of PSA controls with NED controls—to assess markers of PSA recurrence—will be presented in a subsequent paper). SYS cases and PSA controls were matched (1:1) on birth year, calendar year of PSA rise, initial diagnostic pathologic Gleason score (<=6 vs. >=7). The list of eligible cases and controls was scrambled for the blind ascertainment of blocks, isolation of RNA and performance of the expression array experiments.

Table 1A summarizes the distribution of clinical parameters between the SYS cases and the PSA and NED control groups. As expected, there was no significant difference between the groups for the variables used for matching (there was no significant difference in Gleason score when the <=6 and >7 groups—the matching criteria—were compared). Because Gleason scoring may have changed over time, all of the macrodissected lesions were blindly re-graded by a single experienced pathologist (providing a revised Gleason score). As expected, Gleason scores have increased over time. In addition, the proportion of Gleason 8-10 tumors increased comparing NED controls to PSA controls, and PSA controls to SYS cases. Because of this change in grade, the revised Gleason score was used in all the biomarker modeling.

TABLE 1A
Systemic progression (SYS) Case and PSA recurrence (PSA) and no evidence
of disease (NED) control patient demographics

| | Progression group | | | p-value | |
|---|---|---|---|---|---|
| | | | SYS | NED vs. | PSA vs. |
| | NED controls | PSA controls | cases | PSA | SYS |
| Year of surgery | | | | 0.707 | 0.592 |
| N | 213 | 213 | 213 | | |
| Median | 1992 | 1992 | 1992 | | |
| Q1, Q3 | 1989, 1995 | 1990, 1995 | 1989, 1995 | | |
| Age at RRP | | | | 0.682 | 0.496 |
| N | 213 | 213 | 213 | | |
| Median | 67 | 67 | 67 | | |
| Q1, Q3 | 61, 70 | 61, 70 | 61, 70 | | |
| PSA at RRP | | | | 0.001 | 0.957 |
| N | 205 | 208 | 204 | | |
| Median | 8.1 | 10.5 | 10.6 | | |
| Q1, Q3 | 5.1, 13.1 | 6.4, 21.4 | 6.5, 20.7 | | |
| Gleason score, original | | | | 0.411 | 0.024 |
| Missing | 12 | 6 | 14 | | |
| <=6 | 45 (22.4%) | 48 (23.2%) | 46 (23.1%) | | |
| 7 | 139 (69.2%) | 129 (62.3%) | 94 (47.2%) | | |
| 8-10 | 17 (8.5%) | 30 (14.5%) | 59 (29.6%) | | |
| Gleason score, revised | | | | 0.002 | <0.001 |
| Missing | 8 | 2 | 6 | | |
| <=6 | 50 (22.4%) | 32 (15.2%) | 8 (3.9%) | | |
| 7 | 114 (55.6%) | 113 (53.6%) | 75 (36.2%) | | |
| 8-10 | 41 (20.0%) | 66 (31.3%) | 124 (59.9%) | | |
| Stage | | | | 0.138 | <0.001 |
| T2N0 | 118 (55.4%) | 95 (44.6%) | 59 (27.7%) | | |
| T3aN0 | 43 (20.2%) | 53 (24.9%) | 47 (22.1%) | | |
| T3bN0 | 21 (9.9%) | 54 (25.4%) | 56 (26.3%) | | |
| T3xN+ | 31 (14.6%) | 11 (5.2%) | 51 (23.9%) | | |
| Ploidy | | | | 0.525 | 0.001 |
| Missing | 13 | 9 | 1 | | |
| Diploid | 136 (68.0%) | 128 (62.7%) | 97 (45.8%) | | |
| Tetraploid | 53 (26.5%) | 61 (29.9%) | 84 (39.6%) | | |
| Aneuploid | 11 (5.5%) | 15 (7.4%) | 31 (14.6%) | | |
| Age at PSA recurrence | | | | NA | 0.558 |
| N | | 213 | 213 | | |
| Median | | 69.1 | 69.6 | | |
| Q1, Q3 | | 64.2, 73.4 | 64.7, 73.8 | | |

All paraffin-embedded blocks from eligible men were identified, and each block was surveyed for the tissue present (primary and secondary Gleason cancer regions, normal and metastatic lymph nodes, etc.). The dominant Gleason pattern region was macrodissected from the available blocks, and RNA was isolated from that region. Illumina Cancer Panel™ and custom prostate cancer panel DASL array analyses were then performed on all RNA specimens. The Experimental Procedures section and Supplemental Tables 1 & 2 of U.S. Provisional Patent Application No. 61/057,698, filed May 30, 2008, describe the composition of the Cancer Panel and the design of the Custom Panel.

Table 1B summarizes the final block availability, the RNA isolation success rate, and the success rates of the expression array analyses. Of the 639 eligible patients, paraffin blocks were available on 623 (97.5%). Similarly, RNA was successfully isolated and the DASL assays successfully performed on a very high proportion of patients/specimens: Usable RNA was prepared from all 623 blocks, and the Cancer Panel and custom prostate cancer panel DASL arrays were both successful (after repeating some specimens—see below) on 596 RNA specimens (95.7% of RNAs; 93.3% of design patients). Only 9 (1.4%) RNA specimens failed both expression panels. The primary reason for these failures was poor RNA quality—as measured by qRT-PCR of the RPL13A gene expression (Bibikova et al., Genomics, 89(6):666-72 (2007)). Of the 1246 initial samples run on both panels, 87 (7.0%) specimens failed. Those specimens for which there was residual RNA were repeated with a success rate of 77.2% (61 of 79 samples).

TABLE 1B
Availability of blocks, RNA isolation success and DASL assay success

| | Pregression Case/ | | | |
|---|---|---|---|---|
| | Control Group | | | |
| | None | PSA | Systemic | Total |
| Design Number | 213 | 213 | 213 | 639 |
| Blocks Available | 205 | 211 | 207 | 623 (97.5%) |
| Usable RNA | 205 | 211 | 207 | 623 |
| Evaluable Data, Both DASL | 195 | 201 | 200 | 596 (95.7%) |
| Panels | | | | |
| Evaluable Data, Cancer Panel | 3 | 5 | 2 | 10 |
| Evaluable Data, Custom Panel | 2 | 3 | 3 | 8 |
| Failed Both Panels | 5 | 2 | 2 | 9 (1.4%) |

### Expression Analysis Reproducibility

Replicate analysis results, RT-PCR comparisons, and inter- and intra-panel gene expression comparisons are as follows.

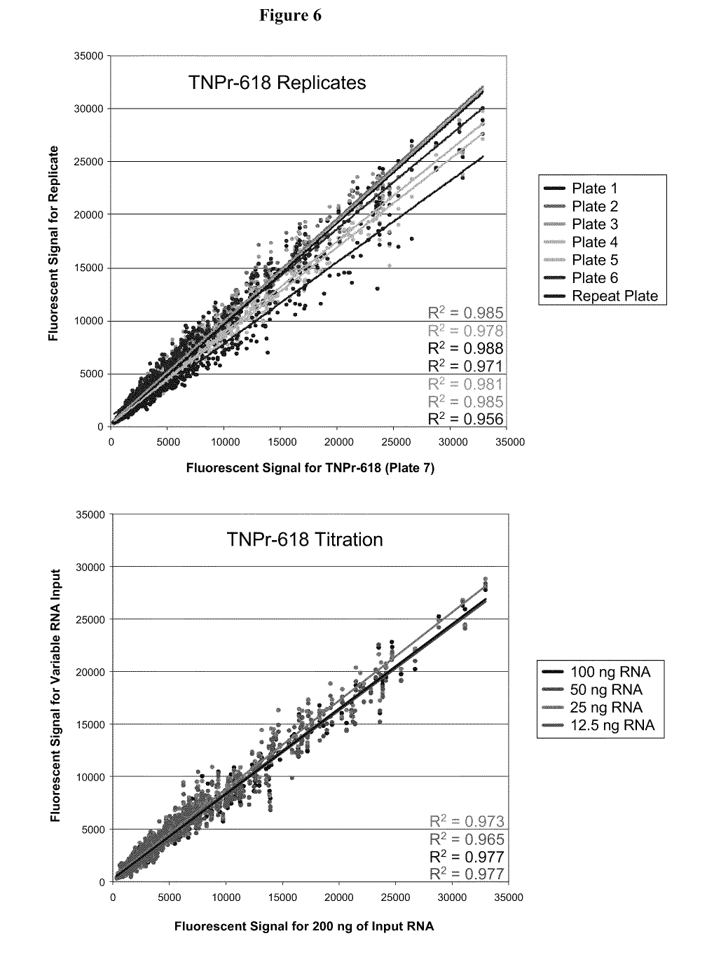

Replicate analyses: The study design included several intra- and inter-run array replicates. To determine inter-run array variability, two specimens were run on each of 8 Cancer Panel v1 array runs. The median (range) inter-run correlation coefficients (r2) comparing these two specimen replicates were 0.94 (0.89-0.95) and 0.98 (0.90-0.98), respectively. The same two specimens were run on each of 8 custom prostate cancer panel array runs. The median (range) inter-run correlation coefficients (r2) comparing these specimen replicates were 0.97 (0.95-0.98) and 0.98 (0.96-0.99), respectively. FIG. 6A summarizes the inter-run replicates for one of the specimens on the custom panel. Twelve specimens were evaluated as intra-run array replicates. The median (range) intra-run r2 values comparing these paired specimens on the Cancer Panel v1 was 0.98 (0.93-0.99). The median (range) intra-run r2 values comparing these paired specimens on the custom panel was 0.98 (0.88-0.99). Two specimens were serially diluted, and the expression results of the diluted RNA specimens compared to that of the standard 200 ng of the parental RNA specimen. The r2 for RNA specimens of 25, 50, and 100 ng ranged from 0.98-0.99 (FIG. 6B) with slopes near 1.0.

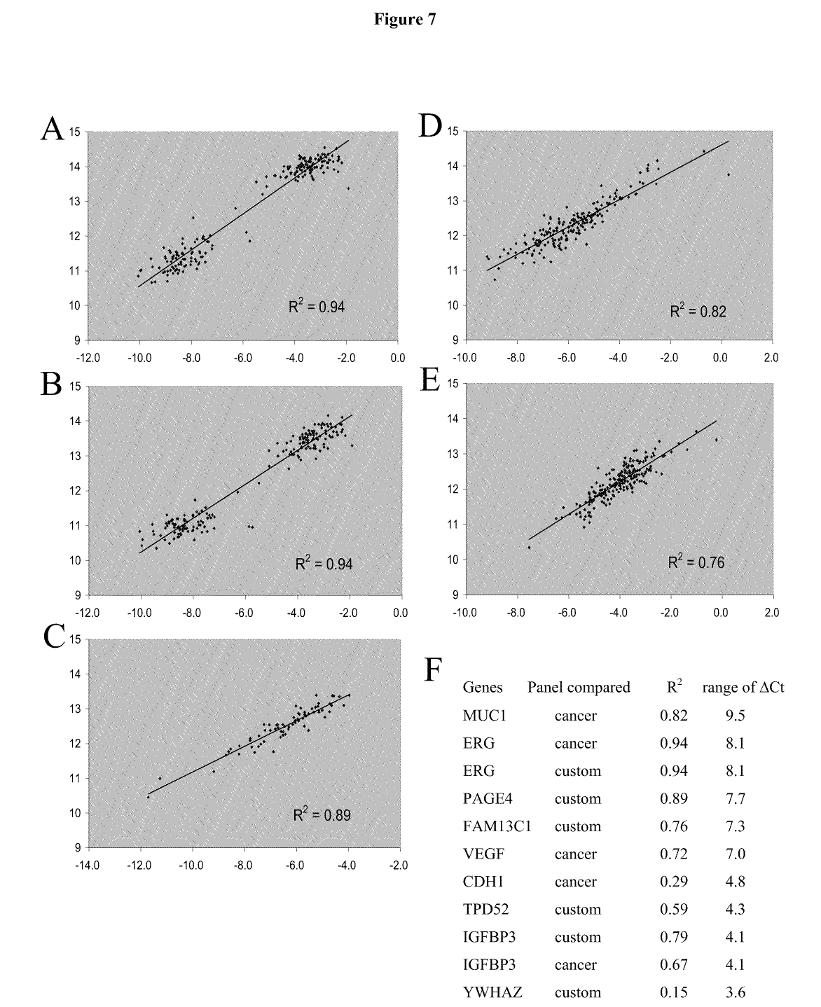

Comparison with RT-PCR: RT-PCR analyses were performed for 9 genes (CDH1, VEGF, MUC1, IGFBP3, ERG, TPD52, YWHAZ, FAM13C1, and PAGE4) on 210 samples. Example results are illustrated in FIG. 7. Comparison of the quantitative RT-PCR and the DASL results gave r2 values of 0.72-0.94 for genes with dynamic range of at least 7 ΔCTs. Genes with a smaller dynamic range of ΔCT gave r2 values of 0.15-0.79 (FIG. 7). Thus, both the DASL and RT-PCR measurements appear to be highly correlated with each other when there is a broad range of RNA expression values.

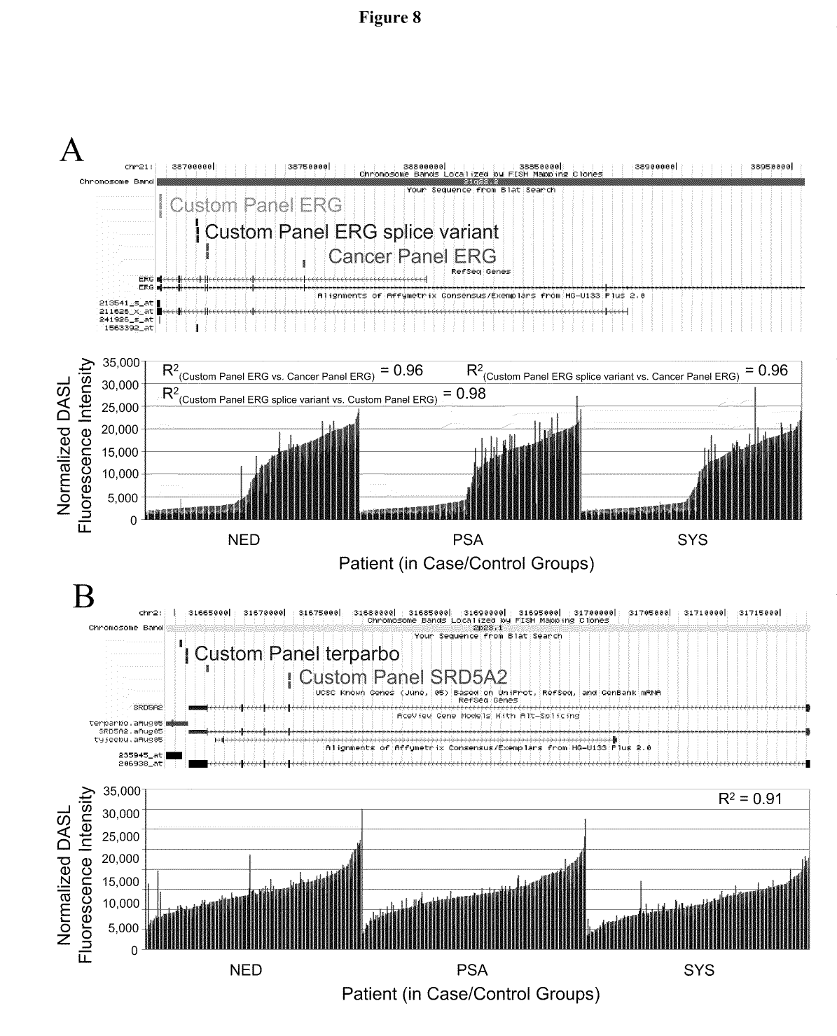

Inter- and Intra-Panel Gene Expression Comparisons: By design several genes were evaluated twice on the custom and/or cancer panels. As an example of a specific inter-panel gene expression comparison, probe sets for ERG were present on both the custom (two 3 probe sets) and cancer (one 3 probe set) panels. The r2 comparing the 2 custom probe sets with the commercial probe set for all 596 patients was 0.96 in both cases (FIG. 8A). As an example of a specific intra-custom panel gene expression comparison are the probe sets for SRD5A2 and terparbo. Terparbo is a “novel” gene which is likely a variant of the SRD5A2 transcript (UCSC browser, http://genome.ucsc.edu). The r2 comparing the two custom probe sets for SRD5A2 and terparbo was 0.91 (FIG. 8B).

Specific Gene Expression Results Comparing the Systemic Progression Cohorts with the PSA Progression and No Evidence of Progression Cohorts:

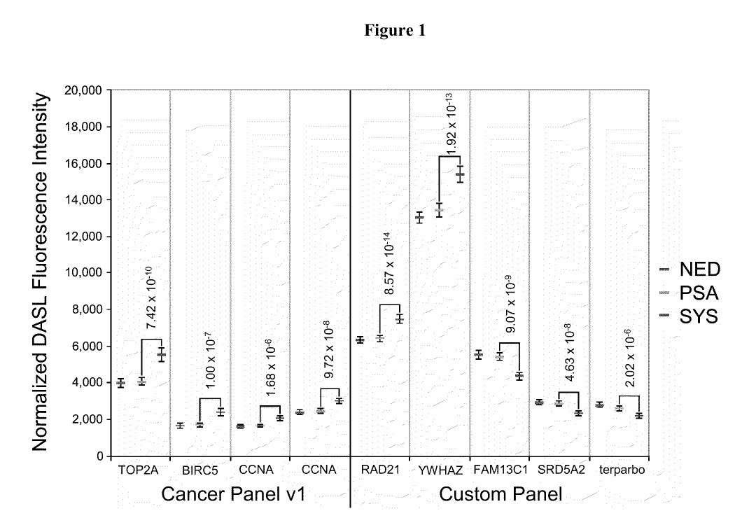

Univariate Analyses by gene: Because the DASL assay appeared to generate precise and reproducible results, the array data was examined for genes whose expression was significantly altered when the SYS cases were compared with the PSA Controls. For this initial analysis, the DASL gene expression value was determined to be the average of the up-to-three probes for each gene on each array. Upon univariate analysis (two-sided t-test) of the probe-averaged and total fluorescence fast-lo normalized data, 68 genes were highly significantly over- or under-expressed in the SYS cases versus PSA controls (p<9.73×10⁻⁷, Bonferroni correction for p<0.001) (Table 2). One hundred twenty-six genes were significantly over- or under-expressed in the SYS cases versus the PSA controls (p<4.86×10⁻⁵, Bonferroni correction for p<0.05). Supplemental Table 3 of U.S. Provisional Patent Application No. 61/057,698, filed May 30, 2008, provides the complete gene list ordered by p-value. FIG. 1 illustrates nine genes with significantly different expression in the SYS cases and PSA controls.

TABLE 2
Top 68 genes highly significantly correlated with prostate cancer systemic
progression (p < 0.001; with Bonferroni correction p < 9.73E−07).

| | | | DASL fast-lo Normalized | | | |
|---|---|---|---|---|---|---|
| | | | Expression Value | | Systemic | Systemic |
| | Gene | Gene | Systemic | PSA | to PSA | to PSA p- |
| Rank | Name | ID* | Progression | Progression | Fold change | value** |
| 1 | RAD21*** | NM_006265 | 7587 | 6409 | 1.18 | 8.57E−14 |
| 2 | YWHAZ | NM_145690 | 15625 | 13417 | 1.16 | 1.92E−13 |
| 3 | TAF2*** | NM_003184 | 3144 | 2681 | 1.17 | 6.99E−13 |
| 4 | SLC44A1 | NM_080546 | 4669 | 4022 | 1.16 | 2.74E−12 |
| 5 | IGFBP3 | NM_000598 | 4815 | 3782 | 1.27 | 3.75E−12 |
| 6 | RHOA | NM_001664 | 15859 | 14542 | 1.09 | 1.22E−11 |
| 7 | MTPN | NM_145808 | 7646 | 6840 | 1.12 | 1.69E−11 |
| 8 | BUB1 | NM_001211 | 1257 | 957 | 1.31 | 2.07E−11 |
| 9 | TUBB | NM_178014 | 17412 | 15659 | 1.11 | 6.52E−11 |
| 10 | CHRAC1*** | NM_017444 | 3905 | 3233 | 1.21 | 6.74E−11 |
| 11 | HPRT1 | NM_000194 | 3613 | 3179 | 1.14 | 8.19E−11 |
| 12 | SEC14L1 | NM_003003 | 7248 | 6185 | 1.17 | 8.20E−11 |
| 13 | SOD1 | NM_000454 | 17412 | 16043 | 1.09 | 1.30E−10 |
| 14 | ENY2 | NM_020189 | 7597 | 6493 | 1.17 | 2.04E−10 |
| 15 | CCNB1 | NM_031966 | 1871 | 1342 | 1.39 | 3.65E−10 |
| 16 | INHBA | NM_002192 | 4859 | 3732 | 1.30 | 5.18E−10 |
| 17 | TOP2A | NM_001067 | 5550 | 4123 | 1.35 | 7.42E−10 |
| 18 | ATP5J | NM_001003703 | 13145 | 11517 | 1.14 | 1.75E−09 |
| 19 | C8orf53*** | NM_032334 | 7373 | 6444 | 1.14 | 1.88E−09 |
| 20 | EIF3S3*** | NM_003756 | 11946 | 10798 | 1.11 | 1.98E−09 |
| 21 | EIF2C2*** | NM_012154 | 5908 | 5338 | 1.11 | 2.12E−09 |
| 22 | CDKN3 | NM_005192 | 1562 | 1229 | 1.27 | 2.32E−09 |
| 23 | TPX2 | NM_012112 | 1193 | 861 | 1.39 | 2.64E−09 |
| 24 | GLRX2 | NM_197962 | 4154 | 3319 | 1.25 | 3.13E−09 |
| 25 | CTHRC1 | NM_138455 | 3136 | 2480 | 1.26 | 3.83E−09 |
| 26 | KIAA0196*** | NM_014846 | 5530 | 4945 | 1.12 | 4.12E−09 |
| 27 | DHX9 | NM_030588 | 7067 | 6607 | 1.07 | 5.02E−09 |
| 28 | FAM13C1 | NM_001001971 | 4448 | 5416 | 0.82 | 9.07E−09 |
| 29 | CSTB | NM_000100 | 16424 | 15379 | 1.07 | 1.57E−08 |
| 30 | SESN3.a | SESN3.a | 8467 | 6811 | 1.24 | 1.99E−08 |
| 31 | SQLE*** | NM_003129 | 2282 | 1832 | 1.25 | 2.43E−08 |
| 32 | IMMT | NM_006839 | 4683 | 4190 | 1.12 | 2.43E−08 |
| 33 | MKI67 | NM_002417 | 4204 | 3261 | 1.29 | 2.91E−08 |
| 34 | MRPL13*** | NM_014078 | 5051 | 4158 | 1.21 | 3.80E−08 |
| 35 | SRD5A2 | NM_000348 | 2318 | 2795 | 0.83 | 4.63E−08 |
| 36 | EZH2 | NM_004456 | 3806 | 3257 | 1.17 | 4.76E−08 |
| 37 | F2R | NM_001992 | 3856 | 3203 | 1.20 | 5.61E−08 |
| 38 | SH3RF2.a | SH3RF2.a | 1394 | 1705 | 0.82 | 6.48E−08 |
| 39 | ZNF313 | NM_018683 | 9542 | 8766 | 1.09 | 7.14E−08 |
| 40 | SDHC | NM_001035511 | 2363 | 2082 | 1.14 | 7.35E−08 |
| 41 | PGK1 | NM_000291 | 2313 | 2001 | 1.16 | 7.84E−08 |
| 42 | GNPTAB | NM_024312 | 5427 | 4587 | 1.18 | 9.04E−08 |
| 43 | meelar.d | meelar.d | 2566 | 3478 | 0.74 | 9.59E−08 |
| 44 | THBS2 | NM_003247 | 3047 | 2458 | 1.24 | 9.72E−08 |
| 45 | BIRC5 | NM_001168 | 2451 | 1802 | 1.36 | 1.00E−07 |
| 46 | POSTN | NM_006475 | 7210 | 5812 | 1.24 | 1.02E−07 |
| 47 | GNB1 | NM_002074 | 12350 | 11206 | 1.10 | 1.20E−07 |
| 48 | FAM49B*** | NM_016623 | 6291 | 5661 | 1.11 | 1.21E−07 |
| 49 | WDR67*** | NM_145647 | 1655 | 1423 | 1.16 | 1.67E−07 |
| 50 | TMEM65.a*** | TMEM65.a | 4117 | 3540 | 1.16 | 1.96E−07 |
| 51 | GMNN | NM_015895 | 7458 | 5945 | 1.25 | 1.99E−07 |
| 52 | PAGE4 | NM_007003 | 6419 | 8065 | 0.80 | 2.00E−07 |
| 53 | MYBPC1 | NM_206821 | 8768 | 11120 | 0.79 | 2.61E−07 |
| 54 | GPR137B | NM_003272 | 3997 | 3447 | 1.16 | 2.96E−07 |
| 55 | ALAS1 | NM_000688 | 5380 | 5035 | 1.07 | 3.55E−07 |
| 56 | MSR1 | NM_002445 | 3663 | 3025 | 1.21 | 3.65E−07 |
| 57 | CDC2 | NM_033379 | 1420 | 1130 | 1.26 | 3.90E−07 |
| 58 | 240093_x_at | 240093_x_at | 1789 | 1469 | 1.22 | 4.71E−07 |
| 59 | IGFBP3 | NM_000598 | 10673 | 9433 | 1.13 | 4.85E−07 |
| 60 | RAP2B | NM_002886 | 3270 | 2922 | 1.12 | 5.00E−07 |
| 61 | MGC14595.a*** | MGC14595.a | 2252 | 1995 | 1.13 | 5.46E−07 |
| 62 | AZGP1 | NM_001185 | 17252 | 20133 | 0.86 | 6.55E−07 |
| 63 | NOX4 | NM_016931 | 2321 | 1942 | 1.19 | 6.67E−07 |
| 64 | STIP1 | NM_006819 | 7630 | 7123 | 1.07 | 7.23E−07 |
| 65 | PTPRN2 | NM_130843 | 4471 | 5398 | 0.83 | 7.36E−07 |
| 66 | CTNNB1 | NM_001904 | 9989 | 9354 | 1.07 | 7.50E−07 |
| 67 | C8orf76*** | NM_032847 | 4088 | 3652 | 1.12 | 7.88E−07 |
| 68 | YY1 | NM_003403 | 9529 | 8635 | 1.10 | 8.08E−07 |

*The Gene ID is the accession number when available. Other Gene IDs can be found by searching the May 2004 assembly of the human genome at http://genome.ucsc.edu/cgi-bin/hgGateway.
**t-test
***Genes mapped to 8q24

### Systemic Progression Prediction Model Development and Testing on Training Set:

The training data were analyzed by panel (cancer, custom and merged), by gene (the average expression for all gene-specific probes), and by individual probes. A statistical model to predict systemic progression (with and without clinical variables) was developed using random forests (Breiman, Machine Learning. 45, 5-32 (2001)) and logistic regression as described herein. Table 3 lists the 15 genes and 2 individual probes selected for the final model.

TABLE 3
Final random forest 17 gene/probe model to predict prostate cancer systemic
progression after a rising PSA following radical prostatectomy

| | | | | Mean DASL Expression Values | | |
|---|---|---|---|---|---|---|
| t-test | | Mean Gini | p-value | Systemic | PSA | Systemic:PSA |
| Rank | Symbol | Decrease* | (t-test) | Progression | Progression | Fold Change |
| 1 | RAD21** | 2.15 | 8.57E−14 | 7587 | 6409 | 1.18 |
| 22 | CDKN3 | 1.28 | 2.32E−09 | 1562 | 1229 | 1.27 |
| 15 | CCNB1 | 1.25 | 3.65E−10 | 1871 | 1342 | 1.39 |
| 12 | SEC14L1 | 1.14 | 8.20E−11 | 7248 | 6185 | 1.17 |
| 8 | BUB1 | 1.06 | 2.07E−11 | 1257 | 957 | 1.31 |
| 55 | ALAS1 | 1.04 | 3.55E−07 | 5380 | 5035 | 1.07 |
| 26 | KIAA0196** | 1.02 | 4.12E−09 | 5530 | 4945 | 1.12 |
| 3 | TAF2** | 1.02 | 6.99E−13 | 3144 | 2681 | 1.17 |
| 78 | SFRP4 | 0.99 | 1.89E−06 | 15176 | 13059 | 1.16 |
| 64 | STIP1 | 0.95 | 7.23E−07 | 7630 | 7123 | 1.07 |
| 25 | CTHRC1 | 0.90 | 3.83E−09 | 3136 | 2480 | 1.26 |
| 4 | SLC44A1 | 0.90 | 2.74E−12 | 4669 | 4022 | 1.17 |
| 5 | IGFBP3 | 0.85 | 3.75E−12 | 4815 | 3782 | 1.27 |
| 307 | EDG7 | 0.82 | 7.07E−03 | 5962 | 6757 | 0.88 |
| 48 | FAM49B** | 0.82 | 1.21E−07 | 6291 | 5661 | 1.11 |
| 19 | C8orf53** | 0.97*** | 1.88E−09 | 7373 | 6444 | 1.14 |
| 275 | CDK10 | 0.53*** | 4.12E−03 | 12254 | 12868 | 0.95 |

*Mean Gini Decrease for a variable is the average (over all random forest trees) decrease in node impurities from recursive partitioning splits on that variable. For classification, the node impurity is measured by the Gini index. The Gini index is the weighted average of the impurity in each branch, with impurity being the proportion of incorrectly classified samples in that branch. The larger the Gini decrease, the fewer the misclassification impurities.
**Genes mapped to 8q24
***Single probes for C8orf53 and CDK10 were selected. The Mean Gini Decrease for these probes are derived from an independent random forest analysis of the all probes separately.

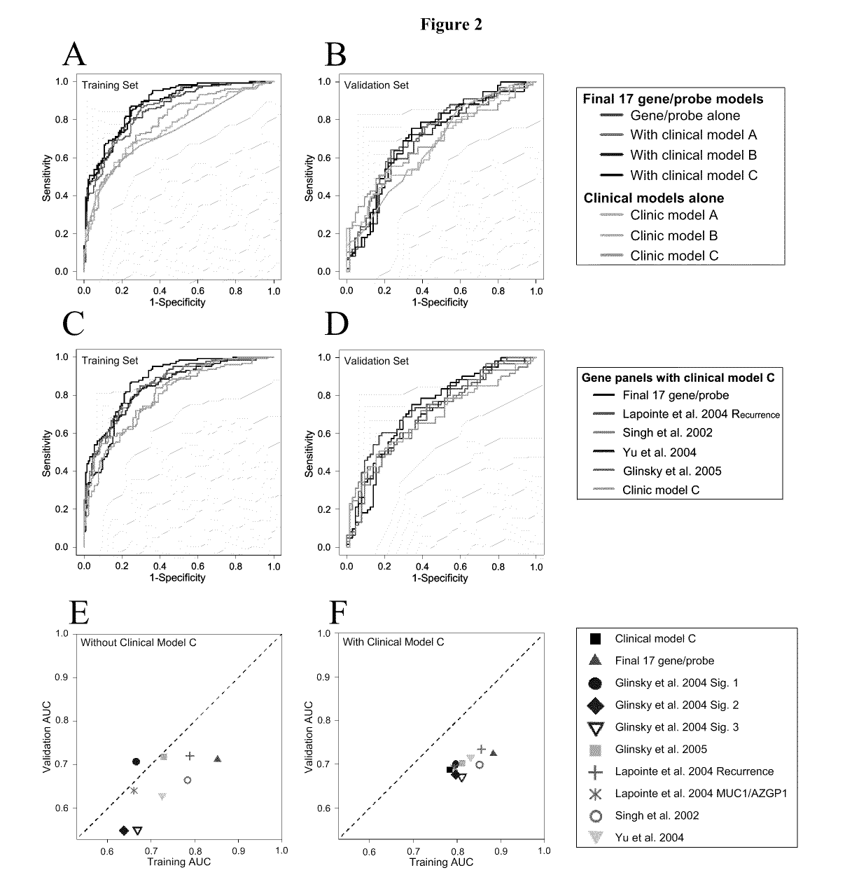

Table 4 and FIG. 2A summarize the areas under the curve (AUCs) for three clinical models, the final 17 gene/probe model and the combined clinical probe models. The variables in the clinical models were those items of clinical information that would be available at specific times in a patient's course. Clinical model A included revised Gleason score and pathologic stage—information available immediately after RRP. The addition of diagnostic PSA and age at surgery did not significantly add to the AUC and was left out of this model. Clinical model B added age at surgery, preoperative PSA value, and any adjuvant or hormonal therapy within 90 days after RRP—information available at RRP after RRP but before PSA recurrence. Clinical model C added age at PSA recurrence, the second PSA level at time of PSA recurrence, and the PSA slope—information available at the time of PSA recurrence.

TABLE 4
Prediction of systemic progression - training set AUCs

| | Probes | Clinical model* | | |
|---|---|---|---|---|
| | alone | A | B | C |
| Clinical model alone | NA | 0.736 | 0.757 | 0.783 |
| Final 17 gene/probe | 0.852 | 0.857 | 0.873 | 0.883 |
| Glinsky et al. 2004 Signature 1 | 0.665 | 0.762 | 0.776 | 0.798 |
| Glinsky et al. 2004 Signature 2 | 0.638 | 0.764 | 0.781 | 0.798 |
| Glinsky et al. 2004 Signature 3 | 0.669 | 0.770 | 0.788 | 0.810 |
| Glinsky et al. 2005 | 0.729 | 0.780 | 0.800 | 0.811 |
| Lapointe et al. 2004 Tumor | 0.789 | 0.825 | 0.838 | 0.855 |
| Recurrence Sig. | | | | |
| Lapointe et al. 2004 (MUC1 and AZGP1) | 0.660 | 0.767 | 0.777 | 0.793 |
| Singh et al. 2002 | 0.783 | 0.824 | 0.838 | 0.851 |
| Yu et al. 2004 | 0.725 | 0.797 | 0.815 | 0.830 |

| | *Clinical model | | |
|---|---|---|---|
| Clinical variable | A | B | C |
| Revised Gleason score | X | X | X |
| pStage | X | X | X |
| Age at surgery | | X | X |
| Initial PSA at recurrence | | X | X |
| Hormone or radiation therapy after RRP | | X | X |
| Age at PSA recurrence | | | X |
| Second PSA | | | X |
| PSA slope | | | X |

A pStage or TNM staging system can be used as described elsewhere (e.g., on the World Wide Web at “upmccancercenters.com/cancer/prostate/TNMsystem.html”).

Using the training set, clinical models A, B and C alone had AUCs of 0.74 (95% CI 0.68-0.80), 0.76 (95% CI 0.70-0.82) and 0.78 (95% CI 0.73-0.84), respectively. The 17 gene/probe model alone had an AUC of 0.85 (95% CI 0.81-0.90). Together with the 17 gene/probe model, clinical models A, B, and C had AUCs of 0.86 (95% CI 0.81-0.90), 0.87 (95% CI 0.83-0.91) and 0.88 (95% CI 0.84-0.92), respectively. A 19 gene model that included the 17 gene/probe model as well as the averaged probe sets for TOP2A and survivin (BIRC5) was tested. Expression alterations have previously been reported to be associated with prostate cancer progression for both genes, and they were included in the top 68 gene list (see Table 2). The addition of these two genes did not improve the prediction of systemic progression in the training set.

The arrays were designed to contain probe sets for several previously published prostate aggressiveness models (Singh et al., 2002, Glinsky et al., 2004, Lapointe et al., 2004, Yu et al., 2004, Glinsky et al., 2005). Table 4 also summarizes the AUCs for array expression results for these models, with and without the inclusion of the three clinical models. FIG. 2C illustrates the AUCs for four of these models with the appropriate comparison with the clinical model C alone and with the 17 gene/probe model. With the clinical data, each of these models generated AUCs that were less than the developed model. However several of the models generated AUCs (e.g. Lapointe et al. 2004 recurrence model, Yu et al. 2004 model, and Singh et al. 2002 model) that were within or close to the 95% confidence limits of our AUC training set estimates.

### Testing of Models on the Validation Set:

The 17 gene/probe model and the other previously published models were then applied to the reserved 205 patient validation set (FIGS. 2B and 2D). FIG. 2E compares the training and validation set AUCs of the each of the gene/probe models alone. With the exception of the Glinsky et al. 2004 Signature 1, all of the gene/probe models had significantly lower AUCs in the validation set compared to the training set. FIG. 2F compares the training and validation set AUCs of each of the gene/probe models including clinical model C. While the 17 gene/probe model and three of the previously published models (LaPointe et al. 2004 Recurrence model, Yu et al. 2004 model and Glinsky et al. 2005 model) outperformed the clinical model alone, the AUCs were significantly lower in the validation set compared to the training set.

The models were compared for their classification of patients into the known PSA progression control and SYS progression case groups. To compare models, the Cramér's V-statistic (Cramér, 1999) was used. Cramér's V-statistic measures how well two models agree. It is calculated by creating a contingency table (2×2 in this case) and computing a statistic from that table. Supplemental Table 4 of U.S. Provisional Patent Application No. 61/057,698, filed May 30, 2008, summarizes the Cramér's V-statistic of the various models, and includes a perfect predictor (“truth”) model for direct evaluation of the models. Briefly, the Cramér's V-statistic ranged from 0.38 to 0.70. The lowest Cramér's V value was between the true state (perfect prediction) and the Glinsky et al. 2005 model with clinical data. The highest Cramér's V value was between our 17 gene/probe model and Singh et al. 2002 model, both with clinical data. Most of the models classified the same patients into the known groups (e.g. classifying a patient in the PSA control group as a PSA progression and a patient in the SYS case group as a systemic progression). They also tended to incorrectly classify the same patients (e.g., classifying a patient in the PSA control group as a systemic progression and vice versa). The 17 gene/probe model correctly classified 5-15 more patients into their known category (PSA controls or SYS cases) compared to the other models.

### Secondary Analyses

Exploratory Survival studies: As noted above, the 17 gene/probe model and the previously reported models each classified some of the SYS cases in the good outcome category (e.g. to be PSA recurrences, not systemic progressors) and some of the PSA controls in the poor outcome category (e.g. to go on to systemic progression). There was a curiosity to see if these apparently false classifications had any biologic or clinical relevance.

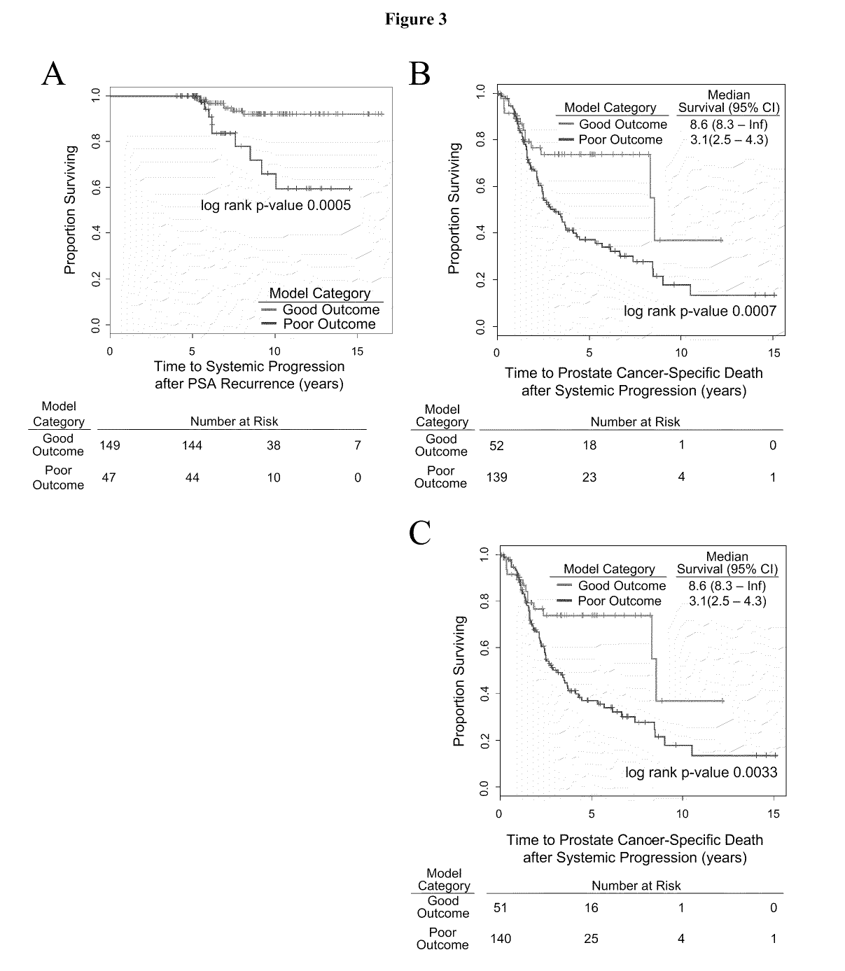

Seventeen men in the PSA control group (who had both array and clinical model C data) went on to have systemic progression beyond 5 years at the time of last follow-up. Of these 17 patients, 9 were predicted to have a poor outcome by the 17 gene/probe model. Of the 179 patients who did not have any systemic progression, 38 were classified in the poor outcome category by the model (p value=0.0066, Fisher exact test). FIG. 3A illustrates the systemic progression-free survival for the good and poor outcome groups in the PSA controls. PSA controls whose tumor classified as having a poor outcome had significantly increased hazard of developing systemic progression beyond 5 years (log rank p-value=0.00050) (HR=4.7, 95% CI: 1.8-12.1).

Ninety-three men in the SYS case group (who also had array and clinical model C data) went on to prostate cancer death at the time of last follow-up. Of these 93 patients, 78 were predicted to have a poor outcome by the 17 gene/probe model. Of the 98 patients who did not suffer a prostate cancer death, 61 were classified in the poor outcome category by the model (p value=0.0008, chi-square test). FIG. 3B illustrates the prostate cancer-specific overall survival for the good and poor outcome groups in the SYS cases. SYS cases whose tumor classified as having a poor outcome had significantly increased hazard of suffering a prostate cancer-specific death (HR=2.5, 95% CI: 1.5-4.4). The median survival from first positive bone scan or CT was 2.8 years (95% CI: 2.4-4.2) in the group classified as having a poor outcome and 8.6 years (95% CI: 7.4-∞) in the group classified as having a good outcome (log rank p-value=0.00068).

Similar associations were observed when 3 of the previously published models with high AUCs (Lapointe et al. 2004 recurrence model and the Glinsky et al. 2005 and Yu et al. 2004 models) were evaluated. The following describes the results for the LaPointe et al. 2004 recurrence model (data for the other two models were similar and not shown). Of the 98 patients who did not suffer a prostate cancer death, 60 were predicted to have a poor outcome by the Lapointe et al. 2004 recurrence model (p value=0.0001, chi-square test). FIG. 3C illustrates the prostate cancer-specific overall survival for the good and poor outcome groups in the SYS cases. SYS cases whose tumor classified as having a poor outcome had significantly increased hazard of suffering a prostate cancer-specific death (HR=2.3, 95% CI: 1.3-4.2). The median survival from first positive bone scan or CT was 3.1 years (95% CI: 2.5-4.3) in the group classified as having a poor outcome and 8.6 years (95% CI: 8.3-∞) in the group classified as having a good outcome (log rank p-value=0.0033).

Exploratory 8q24 Studies: Because of recent tumor chromosome dosage and germ line association studies, the custom array included 82 8q genes on the custom array. Fourteen 8q genes were within the top 68 genes upon univariate analysis (Table 2). Compared to the proportion of 8q gene on both arrays the prevalence of 8q genes is non random (p=0.003, Fisher exact test). Twelve additional 8q genes were within the top 126 genes. The prevalence of 26 8q genes in the top 126 is statistically significant (p=1.56×10-5, Fisher exact test). Chromosome band 8q24.1 has the greatest over-representation of genes in the top 68 gene and 126 gene lists (11 genes, p=6.35×10-7 and 19 genes, p=9.34×10-12, Fisher exact test). Of the 17 genes/probes in our final model, 5 map to 8q24 (p=0.0043, Fisher exact test)(see Table 3).

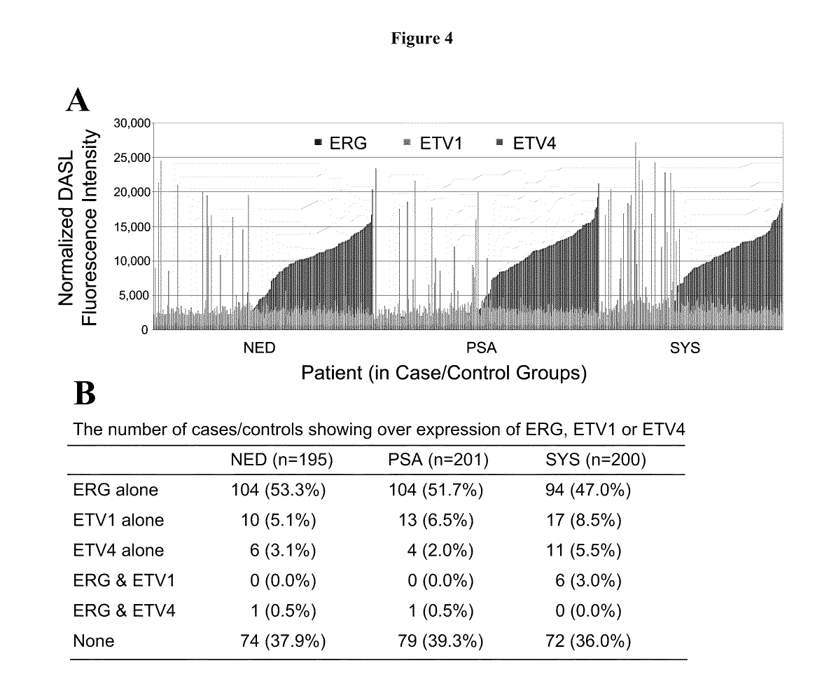

Exploratory ETS Transcription Factor Studies: Alterations of several ETS-family oncogenes are associated with the development of prostate cancer (Tomlins et al., Science. 310, 644-648 (2005); Tomlins et al., Cancer Res. 66, 3396-3400 (2006); and Demichelis et al., Oncogene. 26:4596-4599 (2007)). Oligonucleotide probe sets for the three major members of the ETS family involved in prostate cancer were included: ERG, ETV1, and ETV4, as well as their translocation partner TMPRSS2. FIG. 4 summarizes the expression results for these genes for the SYS cases and the PSA and NED controls. Several observations can be made: 1) With only 8 exceptions ERG, ETV1 and ETV4 overexpression are mutually exclusive; e.g. the overexpression of each generally occurs in different tumors. 2) Different probe sets for ERG give nearly identical expression results (FIG. 8A). 3) The prevalence of ERG overexpression was 50.0%, 52.2% and 53.8% in the SYS cases, PSA controls and NED controls, respectively (using a cutoff of 3200 normalized fluorescence units—see FIG. 4). There is no significant difference in the mean expression and the prevalence of ERG overexpression between the three cohorts. 4) The prevalence of ETV1 overexpression was 11.5%, 6.5% and 5.1% in the SYS cases, PSA controls and NED controls, respectively (using the cutoff of 6000 normalized fluorescence units—see FIG. 4). The prevalence of ETV1 overexpression was significantly higher in SYS Cases (p=0.043, chi-square test). 5) The prevalence of ETV4 overexpression ranged from 2.5%-5.5% among the three groups and was not significantly different. 6) None of the genes were selected by the formal statistical modeling (see Table 3). In fact, the 17 gene/probe model predicted similar rates of progression in ERG+ and ERG− patients.

Exploratory Pathway Analysis: The 461 genes from both cancer and custom panels that are potentially differentially expressed between SYS cases and PSA controls (p≦0.05) were used as the focus genes for Ingenuity Pathway Analysis (IPA, Ingenuity Systems Inc., Redwood City, Calif.). IPA identified 101 canonical pathways that are associated with the focus genes, 51 of which are over-represented with p≦0.05 (see Supplemental Table 5 of U.S. Provisional Patent Application No. 61/057,698, filed May 30, 2008). However, because a limited number of genes on both DASL panels was measured, the p values from IPA analysis may not accurately quantify the degree of over-representation of focus genes in each pathway.

Gene Set Enrichment Analysis (GSEA) (Subramanian et al., Proc Natl Acad Sci USA. 102, 15545-15550 (2005)) was then performed on chromosome 8 genes grouped by map location. Genes mapped to 8q24.1 had a significant p value (p=0.0002) with a FDR q value=0.001 (see Supplemental Table 6 of U.S. Provisional Patent Application No. 61/057,698, filed May 30, 2008).

It was concluded that the measurement of gene expression patterns may be useful for determining which men may benefit from additional therapy after PSA recurrence. These measurements should be included in prospective evaluation of various therapeutic interventions in this setting.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for predicting whether or not a human, at the time of PSA reoccurrence or retropubic radial prostatectomy, will later develop systemic disease, wherein said method comprises:
(a) determining an expression profile score for cancer tissue from said human, wherein said expression profile score is based on at least the expression levels of RAD21, CDKN3, CCNB1, SEC14L1, BUB1, ALAS1, KIAA0196, TAF2, SFRP4, STIP1, CTHRC1, SLC44A1, IGFBP3, EDG7, FAM49B, C8orf53, and CDK10 nucleic acid, and
(b) prognosing said human as later developing systemic disease or as not later developing systemic disease based on at least said expression profile score.

2. The method of claim 1, wherein said method is performed at the time of said PSA reoccurrence.

3. The method of claim 1, wherein said method is performed at the time of said retropubic radial prostatectomy.

4. The method of claim 1, wherein said expression levels are mRNA expression levels.

5. The method of claim 1, wherein said prognosing step (b) comprises prognosing said human as later developing systemic disease or as not later developing systemic disease based on at least said expression profile score and a clinical variable.

6. The method of claim 5, wherein said clinical variable is selected from the group consisting of a Gleason score and a revised Gleason score.

7. The method of claim 5, wherein said clinical variable is selected from the group consisting of a Gleason score, a revised Gleason score, age at surgery, initial PSA at recurrence, use of hormone or radiation therapy after radical retropubic prostatectomy, age at PSA recurrence, the second PSA level at time of PSA recurrence, and PSA slope.

8. The method of claim 1, wherein said method comprises prognosing said human as later developing systemic disease based on at least said expression profile score.

9. The method of claim 1, wherein said method comprises prognosing said human as not later developing systemic disease based on at least said expression profile score.

10. A method for predicting whether or not a human, at the time of systemic disease, will later die from prostate cancer, wherein said method comprises:
(a) determining an expression profile score for cancer tissue from said human, wherein said expression profile score is based on at least the expression levels of RAD21, CDKN3, CCNB1, SEC14L1, BUB1, ALAS1, KIAA0196, TAF2, SFRP4, STIP1, CTHRC1, SLC44A1, IGFBP3, EDG7, FAM49B, C8orf53, and CDK10 nucleic acid, and
(b) prognosing said human as later dying of said prostate cancer or as not later dying of said prostate cancer based on at least said expression profile score.

11. The method of claim 10, wherein said expression levels are mRNA expression levels.

12. The method of claim 10, wherein said prognosing step (b) comprises prognosing said human as later developing systemic disease or as not later developing systemic disease based on at least said expression profile score and a clinical variable.

13. The method of claim 12, wherein said clinical variable is selected from the group consisting of a Gleason score and a revised Gleason score.

14. The method of claim 12, wherein said clinical variable is selected from the group consisting of a Gleason score, a revised Gleason score, age at surgery, initial PSA at recurrence, use of hormone or radiation therapy after radical retropubic prostatectomy, age at PSA recurrence, the second PSA level at time of PSA recurrence, and PSA slope.

15. The method of claim 10, wherein said method comprises prognosing said human as later dying of said prostate cancer based on at least said expression profile score.

16. The method of claim 10, wherein said method comprises prognosing said human as not later dying of said prostate cancer based on at least said expression profile score.

17. A method for (1) predicting whether or not a patient, at the time of PSA reoccurrence, will later develop systemic disease, (2) predicting whether or not a patient, at the time of retropubic radial prostatectomy, will later develop systemic disease, or (3) predicting whether or not a patient, at the time of systemic disease, will later die from prostate cancer, wherein said method comprises determining whether or not cancer tissue from said patient contains an RAD21, CDKN3, CCNB1, SEC14L1, BUB1, ALAS1, KIAA0196, TAF2, SFRP4, STIP1, CTHRC1, SLC44A1, IGFBP3, EDG7, FAM49B, C8orf53, and CDK10 expression profile indicative of a later development of said systemic disease or said death.
